(19) United States
(12) Patent Application Publication
(10) Pub. No.: US 2025/0229031 A1
(43) Pub. Date: Jul. 17, 2025

(54) AUTOINJECTOR WITH FINGER GRIP

(71) Applicant: AG IP HOLDING, LLC, Okemos, MI (US)

(72) Inventor: Patrick Kenneth Powell, Grosse Pointe Park, MI (US)

(21) Appl. No.: 18/854,721

(22) PCT Filed: Apr. 11, 2023
(86) PCT No.: PCT/US2023/018122

§ 371 Date: Oct. 7, 2024

Related U.S. Application Data
(60) Provisional application No. 63/329,627, filed on Apr. 11, 2022.
(60) Provisional application No. 63/342,653, filed on May 17, 2022.
(60) Provisional application No. 63/345,930, filed on May 26, 2022.
(60) Provisional application No. 63/358,334, filed on Jul. 5, 2022.
(60) Provisional application No. 63/358,337, filed on Jul. 5, 2022.
(60) Provisional application No. 63/359,262, filed on Jul. 8, 2022.
(60) Provisional application No. 63/388,405, filed on Jul. 12, 2022.
(60) Provisional application No. 63/389,205, filed on Jul. 14, 2022.
(60) Provisional application No. 63/389,496, filed on Jul. 15, 2022.
(60) Provisional application No. 63/405,173, filed on Sep. 9, 2022.
(60) Provisional application No. 63/416,647, filed on Oct. 17, 2022.
(60) Provisional application No. 63/482,116, filed on Jan. 30, 2023.

(51) Int. Cl. A61M 5/31; A61M 5/20; A61M 5/315; A61M 5/32

(52) U.S. Cl. CPC A61M 5/3137; A61M 5/2033; A61M 5/2053; A61M 5/31505; A61M 5/3204; A61M 2005/2026; A61M 2005/2073; A61M 2005/31508; A61M 2205/8206

(57) ABSTRACT

An autoinjector includes a pump that has a reservoir for holding a medication and an outlet associated with the reservoir for discharging the medication. The autoinjector has a working side at which the medication is deliverable from the pump, and a non-working side opposite the working side. A housing holds the pump and has at least one finger grip proximally located to the working side, in a further embodiment of the foregoing embodiment, the pump includes a button portion operable to mechanically activate an evacuated driver, and the button portion protrudes from the housing on the working side.

### BACKGROUND

An autoinjector is a medical device used to deliver pre-measured doses of medication to a patient. They are designed to make the delivery of medication easier and more convenient. Autoinjectors can be used for a variety of medications, including insulin for the treatment of diabetes, adrenaline for the treatment of anaphylaxis, and methotrexate for the treatment of arthritis. They are particularly useful for patients who have difficulty self-administering medication, such as those with physical disabilities or impaired vision, or for patients who have a needle-phobia.

One type of autoinjector includes a pre-filled syringe, a spring-loaded mechanism, and a needle. The spring-loaded mechanism is designed to push the plunger of the syringe to deliver the medication into the patient's body. There are also needle-free autoinjectors, also known as jet injectors, that operate by using high-pressure streams of medication to penetrate the skin without the use of a traditional needle. Needle-free autoinjectors use a spring-loaded mechanism to create a high-pressure stream of medication that is forced through a small orifice and into the skin. The high velocity of the stream creates a fine mist that penetrates the skin, delivering the medication into the tissue beneath. Needle-free autoinjectors can be easier to use than traditional injection methods, eliminate the risk of needle-stick injuries, and reduce pain and discomfort associated with injections.

Gripping an autoinjector is an important part of using the device properly to ensure safe and effective medication delivery. An autoinjector may have a handle or grip that the patient can hold onto to keep the device steady during injection. In one example, the autoinjector utilizes a “hammer” grip in which the patient wraps their hand in a fist around the syringe body of the autoinjector so that the needle or jet (for needle-free) points downwardly from the bottom of the patient's fist. While mostly effective, for some patients such a grip can make it challenging to properly orient the autoinjector relative to the patient's skin or to firmly grip the autoinjector without slipping during use.

### SUMMARY

An autoinjector according to an example of the present disclosure includes a pump that has a reservoir for holding a medication, an outlet associated with the reservoir for discharging the medication, a working side at which the medication is deliverable from the pump, and a non-working side opposite the working side. A housing holds the pump and has at least one finger grip proximally located to the working side.

In a further embodiment of the foregoing embodiment, the pump includes a button portion operable to mechanically activate an evacuated driver, and the button portion protrudes from the housing on the working side.

In a further embodiment of any of the foregoing embodiments, the evacuated driver is configured to be activated by pressing the at least one finger grip to move the button portion against an injection surface to exert a force on the button portion that causes retraction of the button portion at least partially into the housing, thereby triggering release of the evacuated driver to pressurize the medication to discharge through the outlet.

A further embodiment of any of the foregoing embodiments includes an electronic control powered by an air-activated battery that is exposed to air upon release of the evacuated driver.

In a further embodiment of any of the foregoing embodiments, the at least one finger grip includes a pocket.

In a further embodiment of any of the foregoing embodiments, the at least one finger grip includes a ring.

In a further embodiment of any of the foregoing embodiments, the at least one finger grip includes first and second finger grip that straddle the pump.

In a further embodiment of any of the foregoing embodiments, the pump includes a stopper disposed in the reservoir, a plunger coupled with the stopper, and an evacuated driver is operable to move the plunger and stopper upon activation of the evacuated driver.

In a further embodiment of any of the foregoing embodiments, the evacuated driver defines a pressure differential that stresses a spring device to store a potential energy and, upon release of the pressure differential the spring device releases and moves the plunger and the stopper to discharge the medication through the outlet.

In a further embodiment of any of the foregoing embodiments, the plunger includes a tip portion and retraction of the button portion at least partially into the housing moves the tip portion to pierce the evacuated driver and thereby release the pressure differential.

In a further embodiment of any of the foregoing embodiments, a direction of the pressing is approximately orthogonal to a direction of movement of the plunger.

A further embodiment of any of the foregoing embodiments includes a lock that has an engaged state in which the lock prevents release of the spring device.

In a further embodiment of any of the foregoing embodiments, the stopper includes a first cross-sectional area and the evacuated driver includes a second cross-sectional area that is larger than the first cross-sectional area by a factor of at least 1.5.

In a further embodiment of any of the foregoing embodiments, the outlet includes a needle.

A further embodiment of any of the foregoing embodiments includes a collapsible needle cover on the working side and a needle cover driver coupled with the collapsible needle cover, the needle cover driver operable to move the needle cover from collapsed position to a deployed position shielding the needle.

In a further embodiment of any of the foregoing embodiments, the outlet includes a nozzle.

In a further embodiment of any of the foregoing embodiments, the housing includes at least one leveling bumper on the working side.

In a further embodiment of any of the foregoing embodiments, the finger grip is adjustable in size.

In a further embodiment of any of the foregoing embodiments, the outlet includes a needle, the pump includes a first evacuated driver configured to move the needle from a stored position in which the needle is inside the housing to a deployed position in which the needle protrudes from the working side, and a second evacuated driver configured to pressurize the medication to discharge through the outlet.

A method for using an autoinjector according to an example of the present disclosure includes providing an autoinjector as in any of the foregoing embodiments, inserting at least one finger into the at least one finger grip with a nail-side of the at least one finger facing the non-working side, using the at least one finger to exert a force on the at least one finger grip and thereby depress the working side against the skin of a patient, and activating the pump to discharge the medication from the reservoir and through the outlet into the patient.

A method for using an autoinjector according to an example of the present disclosure includes providing an autoinjector as in any of the foregoing examples. At least the stopper, the reservoir, and the medication are part of a cartridge that is removeable from the housing, wherein the autoinjector has been used to discharge the medication such that the cartridge is spent. The spent cartridge is removed, and a fresh cartridge is inserted into the housing. The fresh cartridge contains additional medication. The evacuated driver is reset such that the autoinjector is operable for a re-use to discharge the additional medication in the fresh cartridge.

In a further example of any of the foregoing embodiments, the autoinjector is re-useable. A method for re-using the autoinjector includes resetting the vacuum of the evacuated driver such that the spring device is stressed, and at least partially filling the reservoir with fresh medication. The autoinjector may be washed and/or otherwise sterilized as part of the method of re-use, such as prior to the resetting of the vacuum. The autoinjector may include a vacuum port connected to the driver and which is used to connect to a vacuum device to reset the vacuum. Alternatively, the outlet of the autoinjector serves as a vacuum port that is connectable to a vacuum device and through which the vacuum is reset.

In a further example of any of the foregoing embodiments, the reservoir is incorporated into a cartridge that is insertable and removeable from the autoinjector. A method for re-using the autoinjector includes removing a spent cartridge from the autoinjector and then inserting a new cartridge into the autoinjector. The new cartridge includes a reservoir that contains additional medication.

The present disclosure may include any one or more of the individual features disclosed above and/or below alone or in any combination thereof.

### BRIEF DESCRIPTION OF THE DRAWINGS

The various features and advantages of the present disclosure will become apparent to those skilled in the art from the following detailed description. The drawings that accompany the detailed description can be briefly described as follows.

FIG. 1 illustrates an autoinjector with a manual pump.

FIG. 2 illustrates the autoinjector of FIG. 1 in use.

FIG. 3 illustrates another example autoinjector with a pump that has an evacuated driver.

FIG. 4 illustrates the autoinjector of FIG. 3 in use.

FIG. 5 illustrates the autoinjector of FIG. 3 upon release of the evacuated driver of the pump.

FIG. 6 illustrates deployment of the needle from the autoinjector.

FIG. 7 illustrates injection of the medication from the autoinjector.

FIG. 8 illustrates deployment of a needle cover to shield the needle upon removal of the autoinjector from the patient's skin.

FIG. 9 illustrates another example autoinjector that is configured for needle-free injection.

FIG. 10 illustrates the autoinjector of FIG. 9 in use.

FIG. 11 illustrates release of the evacuated drive of the autoinjector.

FIG. 12 illustrates the autoinjector during injection.

FIG. 13 illustrates the autoinjector with fingers adjusters and bumpers.

FIG. 14 illustrates the autoinjector with a lock in the form of a balloon that prevents premature release of the evacuated driver.

FIG. 15 illustrates another example autoinjector that has two evacuated drivers, one for needle deployment and another for injection of the medication.

FIG. 16 illustrates the autoinjector of FIG. 15 upon release of the first evacuated driver to deploy the needle.

FIG. 17 illustrates the autoinjector upon release of the second evacuated driver to inject the medication.

FIG. 18 illustrates deployment of a needle cover on the autoinjector.

FIG. 19 illustrates a mini-autoinjector that has a single finger grip.

FIG. 20 illustrates an insertable and removable cartridge.

FIG. 21 illustrates a vacuum resetting of the evacuated drive.

FIG. 22 illustrates the evacuated drive after resetting.

In this disclosure, like reference numerals designate like elements where appropriate and reference numerals with the addition of one-hundred or multiples thereof designate modified elements that are understood to incorporate the same features and benefits of the corresponding elements.

### DETAILED DESCRIPTION

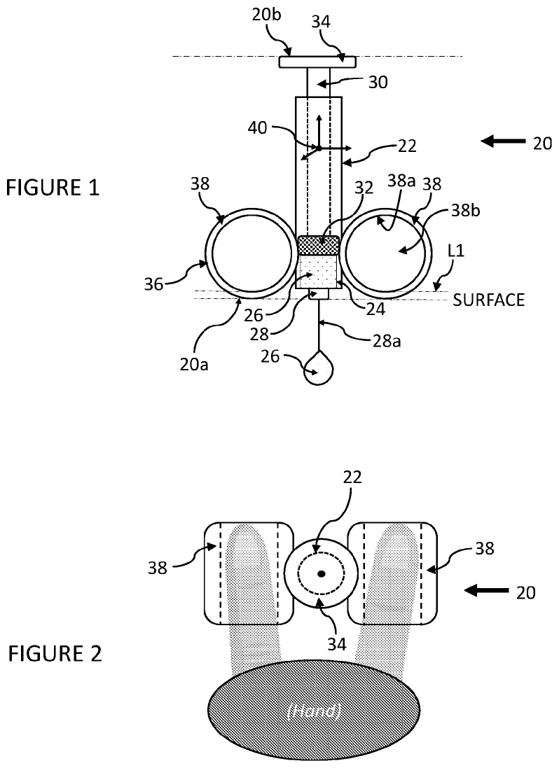

FIG. 1 schematically illustrates an example of an autoinjector 20. The autoinjector 20 may be a single-use device to deliver a pre-measured dose of medication to a patient. Alternatively, the autoinjector 20 may be refilled after a use and then re-used for another injection. As will be appreciated from the examples herein, the autoinjector 20 embodies an ergonomic grip configuration that facilitates use and steady positioning on a patient's skin.

The autoinjector 20 includes a pump 22 that has a reservoir 24 that is pre-filled with a medication 26 in a desired dosage. The medication is a fluid, such as but not limited to, a liquid, a gas, a semi-solid, a gel, a suspension, a flowable powder, a flowable solid (e.g., solid that transitions to liquid form at elevated pressure), or combinations of these. The pump 22 has an outlet 28 associated with the reservoir 24, through which the medication can be dispensed. In this example, the outlet 28 includes a needle 28a, although needle-free configurations are also contemplated. The pump 22 in this example is of a manual syringe type and includes a plunger 30 that has a stopper 32 at one end that is disposed in the reservoir 24 and a handle 34 at its other end protruding from the reservoir 24.

In general, the autoinjector 20 is configured such that it has a working side 20a at which the medication is dispensable from the pump 22 and a non-working side 20b opposite the working side 20a. For example, the working side 20a is the side of the autoinjector 20 that is held against the skin of a patient for injection, and the non-working side 20b is the side of the autoinjector that faces away from the patient's skin. In these regards, the working side 20a may be associated with the bottom-most plane of the autoinjector 20 when in the vertical position, and the non-working side 20b may be associated with the top-most plane of the autoinjector 20 when in the vertical position.

The pump 22 is held in a housing 36. The housing 36 includes at least one finger grip 38 (two shown in this example) proximately located to the working side 20a. The term “proximately located” means that the finger grip 38 is located closer to the working side 20a than to the non-working side 20b. The location of the finger grip 38 is taken with respect to the working surface of the finger grip 38, i.e., the location where pressure is to be applied for use of the autoinjector 20 (for example location L1). In one example, the location is in the first 30% span length of the distance from the working side 20a to the non-working side 20b. In a further example, the location is in the first 15% span length.

In the illustrated example, the finger grips 38 are rings 38a that form pockets 38b for receiving a finger when operating the autoinjector 20, such as the index finger and the middle finger, respectively. The pockets 38b may be smooth, textured, or include a traction material that increases friction to facilitate anti-slipping.

To operate the autoinjector 20, the user inserts a finger into each finger grip 38, as shown in FIG. 2. For instance, the finger or fingers are oriented with the nail-side up, nails facing the non-working side 20b of the autoinjector 20. With the user's palm facing down, the autoinjector 20 is then applied onto the skin at the location of the injection site. The user applies force to the finger grips 38 to press the autoinjector 20 onto the skin, causing the needle 28a to pierce the skin at the injection site. The force applied to the finger grips 38 also helps to keep the autoinjector 20 oriented approximately perpendicular to the skin surface (relative to the plunger movement axis) and facilitates stabilizing the autoinjector 20 from moving or tilting during use. Once in position on the skin, the user presses the handle 34 to move the plunger 30 and stopper 32 against the medication in the reservoir 24. The movement pressurizes the medication 26 and causes it to flow from the reservoir 24, through the outlet 28, and through the needle 28a for injection. Once medication is injected, the user then exerts an opposite, lifting force on the finger grips 38 to lift the autoinjector 20 from the skin surface, thereby also removing the needle 28a from the skin. As will be appreciated, the user will most typically be the patient self-administering the medication. However, the user may alternatively be a clinician, doctor, or other person using the autoinjector 20 to administer the medication to a patient.

The finger grips 38 not only enable the autoinjector 20 to be held against the skin, but the finger grips 38 being proximately located to the working side 20a also provides stability. For instance, by being proximately located to the working side 20a, the finger grips 38 are below the center of gravity 40 (FIG. 1) of the autoinjector 20. By the finger grips 38 being below the center of gravity, movements of the finger grips 38 result in only small movement/alignment changes of the outlet 28 and needle 28a in comparison with a grip location that is above the center of gravity, where the same movement would result in a larger movement/alignment change of the outlet 28 and needle 28a due to the longer distance along the vertical axis of the autoinjector 20. In some instances, a single finger grip 38 may be used, although two finger grips 38 may provide for greater stabilization. For instance, in the depicted example the finger grips 38 straddle the outlet 28/needle 28a, providing a more balanced application of pressure around the injection site.

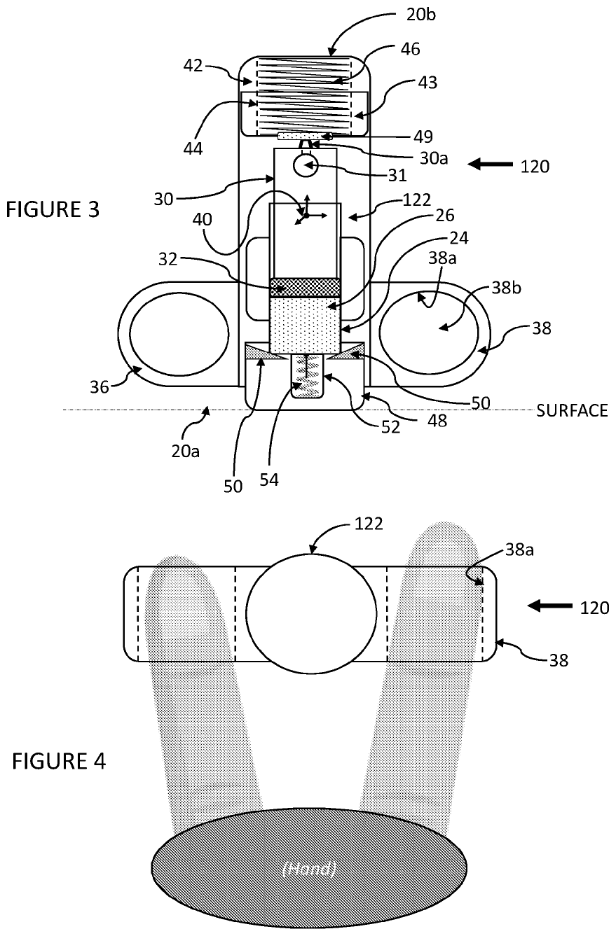

FIG. 3 illustrates another example autoinjector 120 that is similar to the autoinjector 20 but includes a different type of pump 122 that is triggered by application of the autoinjector 120 to the skin rather than by manually pressing the plunger 30. The pump 120 includes an evacuated driver 42 that is operable to move the plunger 30. The evacuated driver 42 includes a casing 44, a spring device 46 in the casing 44, a drive guide 43, and a pierceable seal 49. The casing 44 is expandable and collapsible. In this regard, the casing 44 may include, but is not limited to, an elastically flexible sack, a convoluted sack, a metal foil, or other mechanical structure that permits the casing to expand/collapse. For example, the casing 44 may be of a monolayer or multi-layer construction. The interior of the casing 44 is evacuated and thus defines a pressure differential across the spring device 46, relative to the surrounding ambient environment pressure. That is, the vacuum in the casing 44 relative to the surrounding ambient pressure causes the casing 44 to collapse around, and thus stress, the spring device 46 to provide the stored potential energy. The spring device 46 in the illustrated example is a coil spring. Alternatively, the spring device 46 may include, but is not limited to, a compressible foam, a compressible elastic, a compressible textile, a compressible fluid, a collapsible lattice structure, torsion spring, a constant force spring (i.e., clock spring), or combinations of different types of these or other types of springs, as long as the spring device 46 can be stressed under the vacuum force of the collapsed casing 44 and to store potential energy and then elastically recover to release the potential energy once the pressure differential is equalized. In some instances, the spring device 46 may compress when stressed (e.g., a coil spring or foam), but in other instances the spring device 46 may twist or otherwise deform when stressed (e.g., torsion spring or a constant force spring).

The vacuum in the casing 44 holds the spring device 46 in its stressed, elevated potential energy state (relative to the spring device 46 at rest) until the pressure differential is released. The pressure differential is released by opening the casing 44 to the surrounding ambient environment of the pump 120. Once opened, air enters into the casing 44, thereby equalizing the initial vacuum in the casing 44 with the ambient surroundings. Once equalized, without the vacuum holding the casing 44 in its collapsed state, the potential energy of the spring device 46 is converted to kinetic energy and the spring device 46 expands. Under the force of expansion of the spring device 46, the casing 44 expands to move the plunger 30. The force of release of the spring device 46 may in some configurations be designed to rip or tear the casing 44, rendering it non-reusable, a desirable outcome in some contexts to, for example, eliminate needle sharing among illicit drug users.

To open the casing 44 and release the spring device 46, the pump 120 includes a button portion 48 that initially protrudes from the housing 36 on the working side 20a. The button portion 48 is attached with the reservoir 24 via hinged couplings 50 such that the reservoir 24, the plunger 30, and the stopper 32 are moveable in unison in the retraction direction. Thus, moving the button portion 48 against the injection surface (skin) exerts a force that causes retraction of the button portion 48 at least partially into the housing 36 and pushes the plunger 30 toward the evacuated driver 42. The plunger 30 includes a breaker tip 30a that is situated near the evacuated driver 42. When the button portion 48 retracts, the plunger 30 moves such that the breaker tip 30a pierces the pierceable seal 49 of casing 44 of the evacuated driver 42, thereby triggering release of the evacuated driver 42. A vent passage 31 fluidly connected with the breaker tip 30a that enables air to move freely within the plunger 30 and into the driver 42. The expansion of the spring device 46 then moves the plunger 30, the stopper 32, and the reservoir 24 in the opposite direction toward the working side 20a. The hinged couplings 50 release to decouple the button portion 48 from the plunger 30, stopper 32, and reservoir 24 such that the plunger 30, stopper 32, and reservoir 24 move toward the working side 20a and deploy the needle 28a from the button portion 48 into the skin of the patient. This also causes the plunger 30 to pressurize the medication to discharge through the outlet 28 and needle 28a. Thus, the single evacuated driver 42 deploys the needle 28a and injects the medication.

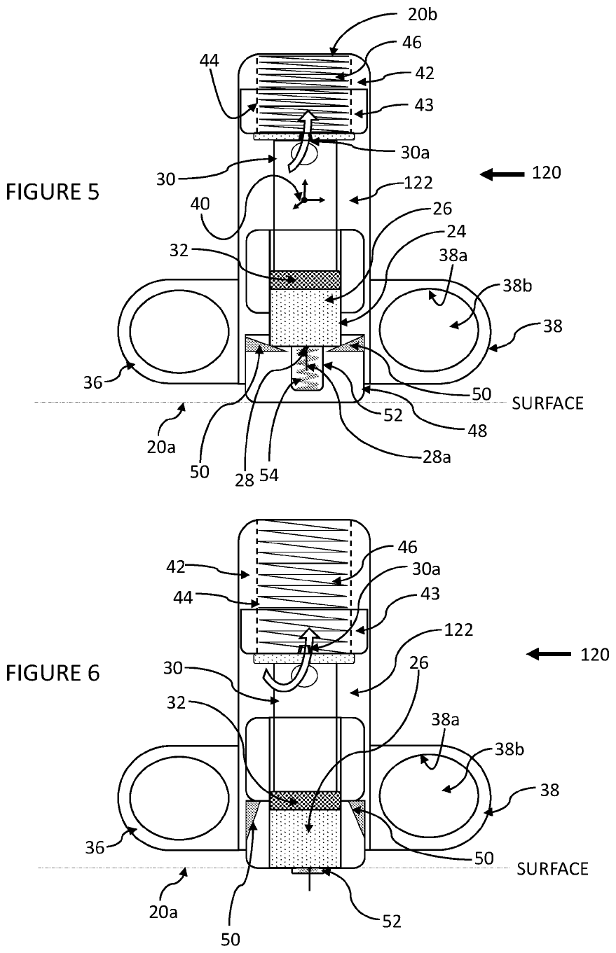

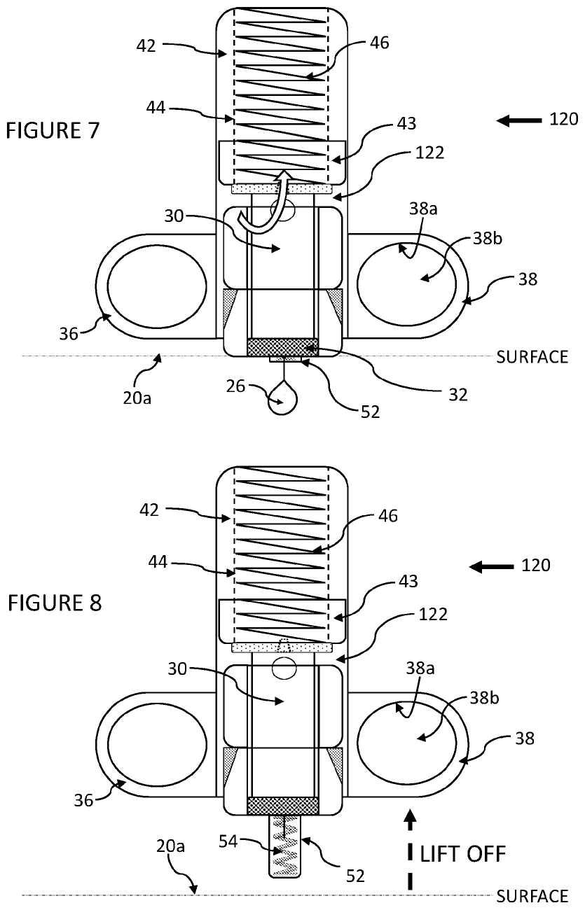

FIGS. 4, 5, 6, and 7, depict operation of the autoinjector 120. Similar to the autoinjector 20, the user inserts a finger into each finger grip 38, as shown in FIG. 4, with fingers oriented nail-side up facing the non-working side 20b of the autoinjector 120. With the user's palm facing down, the autoinjector 120 is then applied onto the skin at the location of the injection site. The user applies force to the finger grips 38 to press the autoinjector 20 onto the skin, as shown in FIG. 5. The applied force causes retraction of the button portion 48 partially into the housing 36 such that the tip 30a pierces the evacuated driver 42. As shown in FIG. 6, expansion of the evacuated driver 42 moves the plunger 30, the stopper 32, and the reservoir 24 toward the working side 20a and thereby deploys the needle 28a to protrude from the button portion 48 into the patient's skin. As depicted in FIG. 7, as the evacuated driver 42 continues to expand, the plunger 30 and stopper 32 pressurize the medication 26 to discharge through the outlet 28 and needle 28a.

FIGS. 3-7 and 8 also depict an optional collapsible needle cover 52 on the working side 20a of the autoinjector 120. The needle cover 52 is attached on the reservoir 24 and circumscribes the needle 28a. The needle cover 52 is coupled with a needle cover driver 54, such as a coil spring under a vacuum. As shown in FIG. 3, the needle cover 52 is initially in a semi-deployed position, shielding and forming a sterile site around the needle 28a. Upon deployment of the needle 28 (FIG. 7), the needle cover 52 is thrust against the skin of the patient, thereby compressing the needle cover 52 and needle cover driver 54 into a collapsed position in which the needle 28a protrudes from the needle cover 52. Upon lifting of the autoinjector 120 from the patient's skin, the needle cover driver 54 decompresses and thereby causes the needle cover 52 to fully deploy from the collapsed position to the fully deployed position shielding the needle 28a, to reduce risk of inadvertent poking.

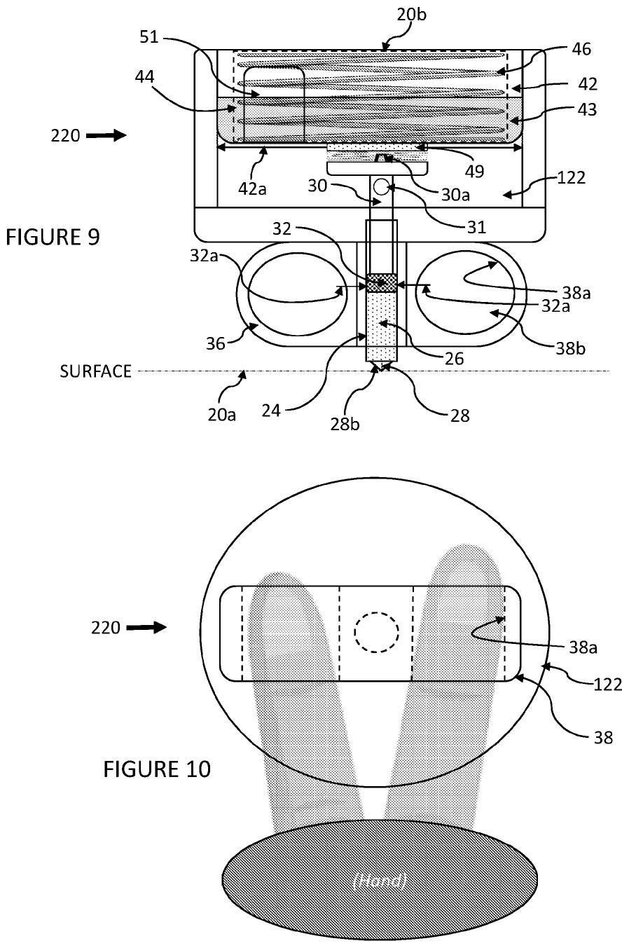

FIG. 9 illustrates another example autoinjector 220 that is similar to the autoinjector 120 but is configured for needle-free injection. In general, needle-free injection requires a high injection force. In this regard, relative to one another, the evacuated driver 42 is large in areal size and the stopper 32 is small in areal size. For instance, the stopper 32 includes a first cross-sectional area represented at 32a and the evacuated driver 42 includes a second cross-sectional area 42a that is larger than the first cross-sectional area by a factor of at least 1.5 and up to a factor of 100 or more. The small cross-sectional area of the stopper 32 relative to the cross-sectional area of the driver 42 acts to increase the pressure inside the reservoir 24 which, acting on the medication inside, injects the medication at high speed/pressure. The outlet 28 may also include a nozzle 28b that is reduced in size to further increase the speed/pressure of the medication. The nozzle 28b can be of a single or plural configuration. Thus, there are two area reductions, that of the driver 42 to the stopper 32 and that of the stopper 32 to the outlet 28, that serve as pressure-elevators.

In further examples, the outlet 28, nozzle 28b, reservoir 24, stopper 32, and stem 30 and may in part or in whole form a replaceable, pre-fillable cartridge or prefilled vial 53 which slides into the main body 36. The controller 51 may include an air-activated battery (e.g., an air-activated zinc-air battery) and electrical circuitry. Upon release of the evacuated drive 42, air entering the drive 42 activates the battery to power the electrical circuitry. The controller 51 may be used for communication to a remote device, such as a cellular phone, to transmit data indicative of use of the autoinjector 220. In this way, drug delivery can be verified, in one example, or, in another example, measure and communicate biometrics of the patient while maintaining battery life during transport. The vacuum across the driver 42 can also be reset, allowing the autoinjector 220 to be re-used. By resetting the evacuated drive 42 and inserting a fresh cartridge, the device can be reusable for delivering multiple pre-filled injections. As will be described in a further embodiment later on below, the resetting of the evacuated drive 42 may be accomplished by an external, vacuum generating device 55 that connects to the nozzle 28 or other port 57 that is in communication with the evacuated drive 42.

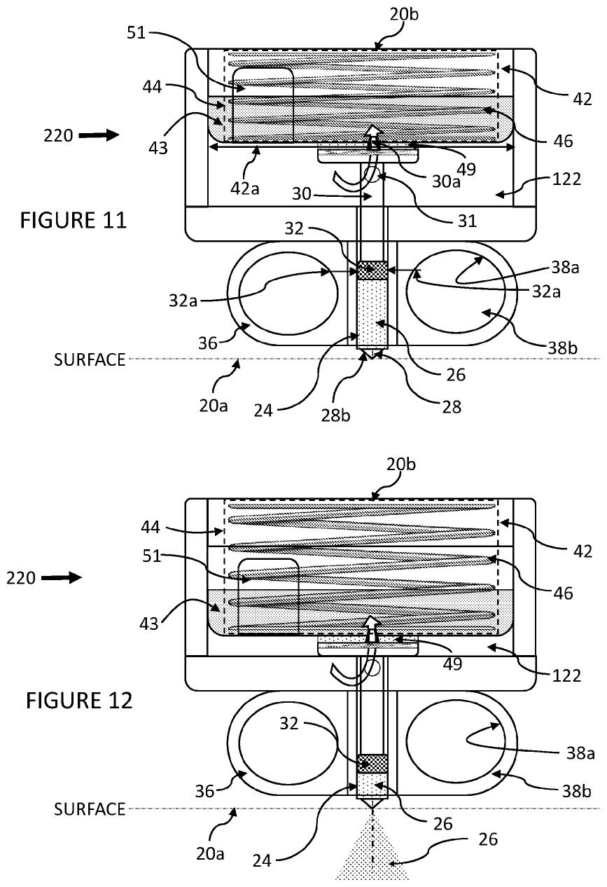

Similar to the autoinjector 20, the user inserts a finger into each finger grip 38, as shown in FIG. 10, with fingers-oriented nail-side up facing the non-working side 20b of the autoinjector 220. With the user's palm facing down, the autoinjector 220 is then applied onto the skin at the location of the injection site. As shown in FIG. 11, the user applies force to the finger grips 38 to press the autoinjector 220 onto the skin. The applied force causes retraction of the nozzle 28b such that the tip 30a pierces the evacuated driver 42. As shown in FIG. 12, expansion of the evacuated driver 42 moves the plunger 30 and stopper 32 to pressurize the medication 26 to discharge through the outlet 28 and nozzle 28b.

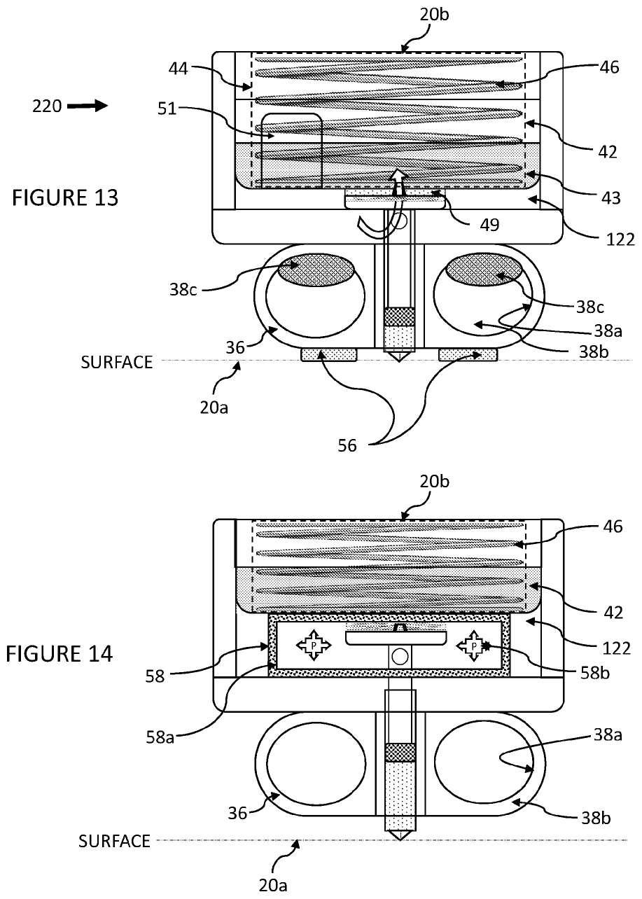

FIG. 13 depicts the autoinjector 220 with additional features that may also be used in any of the other autoinjectors disclosed herein. For instance, each finger grip 38 of the autoinjector 220 includes a finger adjuster 38c configured to change the size of the finger pocket and thereby accommodate different finger sizes. In one example, the finger adjuster 38c is an open cell foam, such as but not limited to, a polyurethane foam that covers a portion of the grip 38 engagement area. When a finger or fingers is/are inserted into the finger grip 38, the open cell foam compresses, enabling engagement with a wide range of finger sizes while maintaining a positive engagement between the finger grip 38 and the fingers for proper operation of the autoinjector 220. As also shown, the autoinjector 220 includes at least one bumper 56 on the working side 20a. The bumper 56 may also be made of an open cell foam, such as the polyurethane foam. The bumper 56 compresses against the patient's skin and facilitates leveling the autoinjector 220 to align the nozzle 28b to a desired injection angle and may also facilitate comfort of use.

FIG. 14 illustrates the autoinjector 220 with a lock 58. As shown, the lock 58 is in an engaged state in which the lock 58 prevents release of the spring device 46 of the evacuated drive 42. Thus, even if the evacuated driver 42 inadvertently releases, the lock 58 prevents discharge of the medication 26 by preventing the spring device 46 from expanding the move the plunger 30 and stopper 32. For example, the lock 58 includes a balloon 58a that is pressurized with a gas in the engaged state, i.e., inflated. The balloon 58a, when in the engaged state, presses against the evacuated drive 42 with a force that exceeds the spring force of the spring device 46 such that if the driver 42 releases the balloon 58a will hold the spring device 46 in its stressed state. The balloon 58a includes one or more release valves 58b. Upon triggering the release valve 58b, which may include a sharp tip to pierce and pop the balloon 58a, the balloon 58a deflates. Thereafter, with the balloon 58a no longer pressing against the evacuated driver 42, the spring device 46 is enabled to release upon activation of the evacuated drive 42. For instance, the lock 58 is utilized as a security feature to prevent premature discharge of the medication, and the user may deactivate the lock 58 to ready the autoinjector 220 for use. The lock 58 may serve as an impact damper between the moving shuttle 43 and the housing 36 during an injection event.

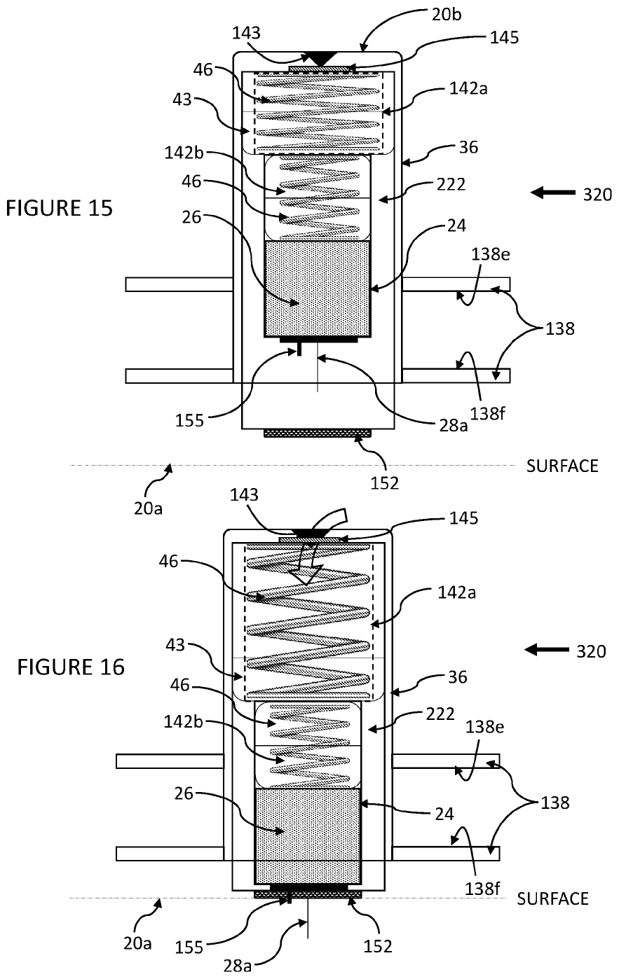

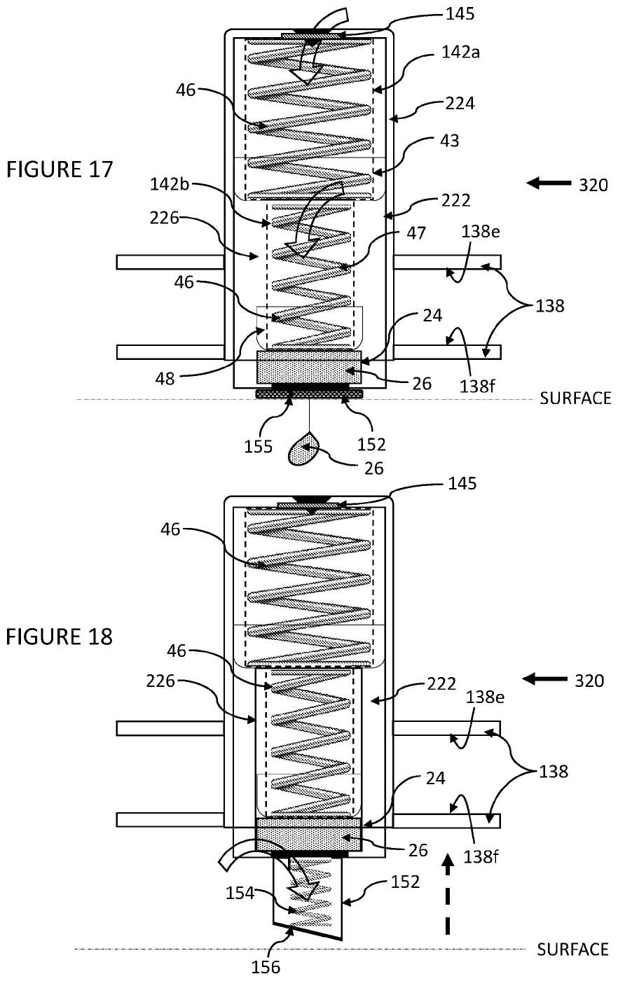

FIG. 15 illustrates another example autoinjector 320 that is similar to the autoinjector 120 but includes a different type of pump 222. The pump 222 includes first and second evacuated drivers 142a/142b. Each driver 142a/142b is configured the same as the afore-described driver 42 (i.e., includes a casing and spring device). The first evacuated driver 142a is configured to move the second evacuated driver 142b, the reservoir 24, and the needle 28a toward the working side 20a. In that regard, the needle 28a moves from a stored position in which the needle 28a is inside the housing 36 to a deployed position in which the needle 28a protrudes at the working side 20a. For instance, the first evacuated driver 142a includes a breaker 143 on the non-working side 20b that the user actuates to pierce the driver 142a at pierceable seal 145 thereby causing the driver 142a to expand, as shown in FIG. 16. The expansion of the first evacuated driver 142a, coupled with resistance of pushing the autoinjector 320 against the skin, causes the first evacuated driver 142a to pierce the second evacuated driver 142b, as shown in FIG. 17. Upon release, the second evacuated driver 142b expands to pressurize the medication to discharge through the outlet 28 and needle 28a.

In this example, the autoinjector 320 also includes a needle cover 152 and needle cover driver 154. The needle cover driver 154 is an evacuated driver similar to the afore-mentioned evacuated drivers. There is a breaker 155 near the needle 28a such that, upon deployment of the needle 28a, the breaker 155 pierces the needle cover driver 154. Upon initial release, however, the needle cover driver 154 is substantially prevented from expanding because it is pressed against the skin of the patient. As shown in FIG. 18, once the needle 28a and autoinjector 320 are removed from the patient, the needle cover driver 154 can then expand and thereby move the needle cover 152 to a deployed position in which it shields the needle 28a, to reduce risk of inadvertent poking. The needle cover 152 may include a rigid face 156 such that once the needle cover 152 deploys the rigid face 156 prevent the needle 28a from poking back through the end face after use. Thus, during use of the autoinjector 320, the needle 28a is only exposed when penetrating the skin, as it is inside the autoinjector 320 prior to deployment and is shielded by the needle cover 152 upon retraction of the needle 28a from the skin.

FIGS. 15-18 also demonstrate another example finger grip 138. In this case, rather than rings, the finger grips 138 each include upper and lower flanges 138e/138f that are spaced apart. The region in between the flanges 138e/138f provide a pocket for receiving the user's finger. Such a configuration may facilitate use for users that have dexterity difficulties wherein more than one finger may be used on one side of the device.

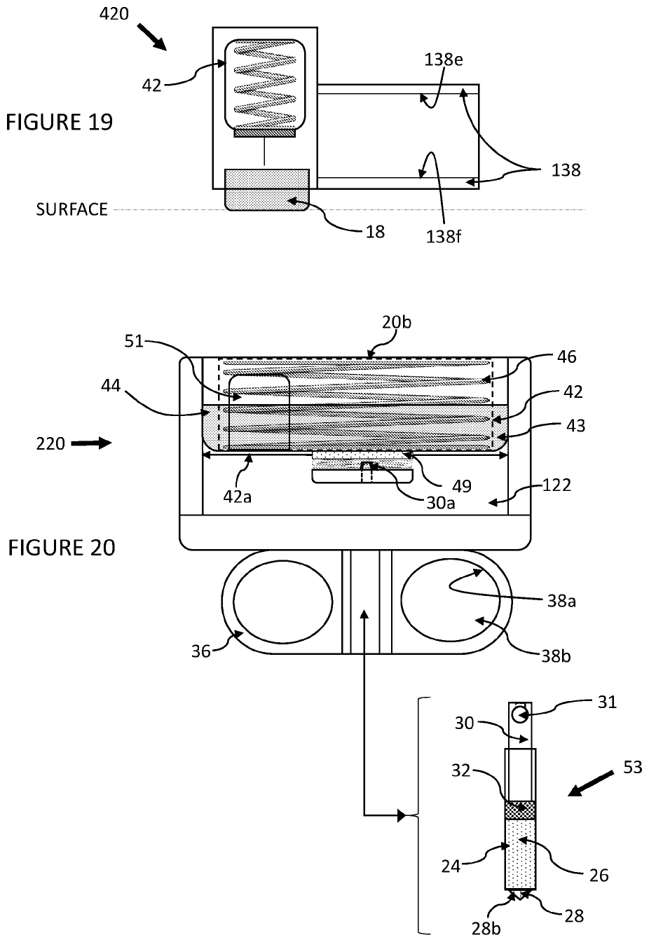

FIGS. 19 illustrates another example autoinjector 420 that is the same as the injector 120 (FIG. 3) but is miniaturized and has a single finger grip 138. In this case, as the autoinjector 420 is small, the single finger grip 138 on one side may be sufficient for balance and proper leveling of the device during use.

FIG. 20 illustrates the autoinjector 220 with a replaceable cartridge 53. The cartridge 53 at least includes the reservoir 24, the stopper 28, and the medication 26 in the reservoir 24, but may also include the nozzle 28, the nozzle orifice 28b, the plunger 30, and vent passage 31. The cartridge 53 can be inserted into the autoinjector 220 and then removed from the autoinjector 220 after use.

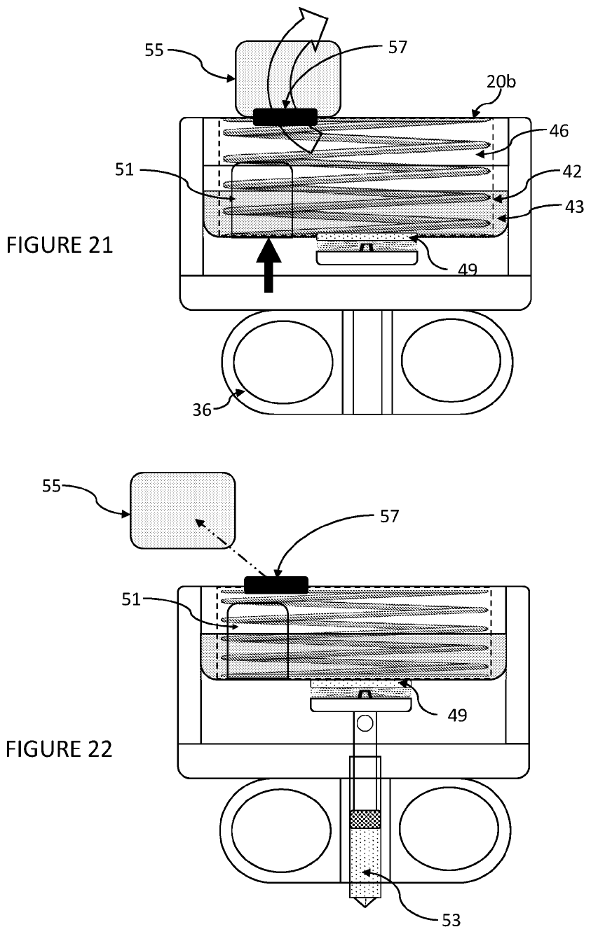

FIG. 21 depicts a resetting of the evacuated drive 42 of the autoinjector 220 by use of an external vacuum pump 55 through a port 57 on the drive 42. For instance, after a use of the autoinjector 220 the initial vacuum in the casing 44 becomes equalized and the autoinjector 220 is thus inoperable for a further use unless the vacuum is reset. In that regard, the vacuum pump 55 is connected to the casing 44 via the port 57 such that the vacuum in the casing 44 is restored to the same or substantially same level as the initial vacuum. Thereafter, as shown in FIG. 22, the vacuum pump 55 can be removed and the port 57 closed before inserting a new cartridge 53 that has additional medication. The autoinjector 220 is then ready for reuse.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. An autoinjector comprising:
a pump including a reservoir for holding a medication and an outlet associated with the reservoir for discharging the medication;
a working side at which the medication is deliverable from the pump, and a non-working side opposite the working side;
a housing holding the pump, the housing having at least one finger grip proximally located to the working side.

2. The autoinjector as recited in claim 1, wherein the pump includes a button portion operable to mechanically activate an evacuated driver, and the button portion protrudes from the housing on the working side.

3. The autoinjector as recited in claim 2, wherein the evacuated driver is configured to be activated by pressing the at least one finger grip to move the button portion against an injection surface to exert a force on the button portion that causes retraction of the button portion at least partially into the housing, thereby triggering release of the evacuated driver to pressurize the medication to discharge through the outlet.

4. The autoinjector as recited in claim 2, further comprising an electronic control powered by an air-activated battery that is exposed to air upon release of the evacuated driver.

5. The autoinjector as recited in claim 1, wherein the at least one finger grip includes a pocket.

6. The autoinjector as recited in claim 1, wherein the at least one finger grip includes a ring.

7. The autoinjector as recited in claim 1, wherein the at least one finger grip includes first and second finger grip that straddle the pump.

8. The autoinjector as recited in claim 1, wherein the pump includes a stopper disposed in the reservoir, a plunger coupled with the stopper, and an evacuated driver is operable to move the plunger and stopper upon activation of the evacuated driver.

9. The autoinjector as recited in claim 8, wherein the evacuated driver defines a pressure differential that stresses a spring device to store a potential energy and, upon release of the pressure differential the spring device releases and moves the plunger and the stopper to discharge the medication through the outlet.

10. The autoinjector as recited in claim 8, wherein the plunger includes a tip portion and retraction of the button portion at least partially into the housing moves the tip portion to pierce the evacuated driver and thereby release the pressure differential.

11. The autoinjector as recited in claim 10, wherein a direction of the pressing is approximately orthogonal to a direction of movement of the plunger.

12. The autoinjector as recited in claim 9, further comprising a lock that has an engaged state in which the lock prevents release of the spring device.

13. The autoinjector as recited in claim 9, wherein the stopper includes a first cross-sectional area and the evacuated driver includes a second cross-sectional area that is larger than the first cross-sectional area by a factor of at least 1.5.

14. The autoinjector as recited in claim 1, wherein the outlet includes a needle.

15. The autoinjector as recited in claim 14, further comprising a collapsible needle cover on the working side and a needle cover driver coupled with the collapsible needle cover, the needle cover driver operable to move the needle cover from collapsed position to a deployed position shielding the needle.

16. The autoinjector as recited in claim 1, wherein the outlet includes a nozzle.

17. The autoinjector as recited in claim 1, wherein the housing includes at least one leveling bumper on the working side.

18. The autoinjector as recited in claim 1, wherein the finger grip is adjustable in size.

19. The autoinjector as recited in claim 1, wherein the outlet includes a needle, the pump includes a first evacuated driver configured to move the needle from a stored position in which the needle is inside the housing to a deployed position in which the needle protrudes from the working side, and a second evacuated driver configured to pressurize the medication to discharge through the outlet.

20. A method for using an autoinjector, the method comprising:
providing an autoinjector that has a pump that includes a reservoir that holds a medication and an outlet associated with the reservoir for discharge of the medication therefrom, a housing that holds the pump, the housing has a working side at which the medication is deliverable from the outlet, a non-working side opposite the working side, and at least one finger grip proximally located to the working side;
inserting at least one finger into the at least one finger grip with a nail-side of the at least one finger facing the non-working side;
using the at least one finger to exert a force on the at least one finger grip and thereby depress the working side against the skin of a patient; and
activating the pump to discharge the medication from the reservoir and through the outlet into the patient.

21. A method for using an autoinjector, the method comprising:
providing an autoinjector that has a pump that includes a reservoir that holds a medication, a plunger that has a stopper that is disposed in the reservoir, an outlet associated with the reservoir for discharge of the medication therefrom, an evacuated driver that is operable to move the plunger to discharge the medication, a housing that holds the pump, the housing has a working side at which the medication is deliverable from a nozzle on the outlet, a non-working side opposite the working side, and at least one finger grip proximally located to the working side,
wherein at least the stopper, the reservoir, and the medication are part of a cartridge that is removeable from the housing, and wherein the autoinjector has been used to discharge the medication such that the cartridge is spent;
removing the spent cartridge;
inserting a fresh cartridge into the housing, wherein the fresh cartridge contains additional medication; and
resetting the evacuated driver such that the autoinjector is operable for a re-use to discharge the additional medication in the fresh cartridge.
